(19) United States
(12) Patent Application Publication
(10) Pub. No.: US 2024/0164842 A1
(43) Pub. Date: May 23, 2024

(54) UTILIZATION OF THREE-DIMENSIONAL NAVIGATION TECHNOLOGY DURING LUNG DENERVATION PROCEDURES

(71) Applicant: Nuvaira, Inc., Minneapolis, MN (US)

(72) Inventors: Philip Johnson, Plymouth, MN (US); Ryan Kaveckis, Minneapolis, MN (US); Larry Wales, Maplewood, MN (US)

(21) Appl. No.: 18/516,008

(22) Filed: Nov. 21, 2023

Related U.S. Application Data
(60) Provisional application No. 63/384,557, filed on Nov. 21, 2022.

(51) Int. Cl. A61B 34/10; A61B 18/00; A61B 18/12; A61B 18/14; A61B 34/00; A61B 34/20

(52) U.S. Cl. CPC A61B 34/10; A61B 18/1206; A61B 18/1492; A61B 34/20; A61B 34/25; A61B 2018/00011; A61B 2018/0022; A61B 2018/00541; A61B 2018/00577; A61B 2018/00648; A61B 2018/00708; A61B 2018/00779; A61B 2018/00791; A61B 2018/00827; A61B 2018/00839; A61B 2018/00863; A61B 2018/00982; A61B 2034/107; A61B 2034/2055; A61B 2034/2065

(57) ABSTRACT

A pulmonary treatment system for treatment of target tissue in an airway of a subject, including a catheter assembly comprising an ablation assembly or tip including an expandable member, at least one energy emitter, and a cooling member, wherein the ablation tip is configured to be positioned within the airway of the subject such that expansion of the expandable member enables the at least one energy emitter and cooling member to engage a wall of the airway, the cooling member configured to cool a portion of the surface of the wall of the airway to reduce damage to the airway disposed between the at least one energy emitter and the target tissue, while the at least one energy emitter delivers energy to the target tissue to create one or more nerve attenuating lesions, and a control assembly including a display configured to depict a three-dimensional graphical rendering of the airway of the subject for preplanning prior to the procedure, with real-time tracking of the ablation tip during the treatment of the target tissue.

### RELATED APPLICATIONS

The present disclosure claims the benefit of U.S. Provisional Application No. 63/384,557, filed Nov. 21, 2022. The complete disclosure of which is hereby incorporated by reference.

### TECHNICAL FIELD

The present disclosure relates generally to pulmonary treatment systems and methods, and more particularly to enabling enhanced, real-time, three-dimensional visualization during application of monopolar or bipolar radiofrequency (RF) ablation to treat target regions within an inside of an airway wall for the purpose of attenuating nerve signals to or from portions of the airway wall.

### BACKGROUND

Pulmonary diseases are some of the most common medical conditions, affecting tens of millions of people in the U.S. alone. Pulmonary diseases result from problems in the respiratory tract that interfere with proper respiration. Many of these diseases require medical attention or intervention in order to restore proper lung function and improve a patient's overall quality of life. Some of the more common pulmonary diseases include asthma and chronic obstructive pulmonary disease or COPD. Symptoms of pulmonary disease like COPD and asthma vary but often include a persistent cough, shortness of breath, wheezing, chest tightness, and breathlessness. Generally, these symptoms are exacerbated when performing somewhat strenuous activities, such as running, jogging, brisk walking, etc. However, these symptoms may be noticed when performing non-strenuous activities if the disease is allowed to progress unchecked. Over time, especially if medical attention is not sought, a person's daily activities will be significantly impaired, thus reducing an overall quality of life.

Many pulmonary diseases, whether acute or chronic, often involve pathologic conditions associated with airway inflammation. When such inflammation has developed at the airway, infiltrated inflammatory cells cause damage to bronchial or lung tissue, which eventually results in the respiratory dysfunction characteristic of pulmonary diseases, such as reduction in respiratory flow rate or oxygen exchange capacity. Over time, this inflammation can lead to blockage of the airway lumen, thickening of the airway wall, and alteration of structures within or around the airway wall. Airway obstruction can significantly decrease the amount of gas exchanged in the lungs resulting in breathlessness. Blockage of an airway lumen can be caused by excessive intraluminal mucus, edema fluid, or both. Thickening of the airway wall may be attributable to excessive contraction of the airway smooth muscle, airway smooth muscle hypertrophy, mucous glands hypertrophy, inflammation, edema, or combinations of these. Alteration of structures around the airway, such as destruction of the lung tissue itself, can lead to a loss of circumferential traction on the airway wall and subsequent narrowing of the airway. Generally, pulmonary diseases like COPD and asthma are the result of a complex interplay of local inflammatory cytokines, inhaled irritants (e.g., cold air, smoke, allergens, or other chemicals), systemic hormones (e.g., Cortisol and epinephrine), local nervous system input (i.e., nerve cells contained completely within the airway wall that can produce local reflex stimulation of smooth muscle cells and mucous glands), and the central nervous system input (i.e., nervous system signals from the brain to smooth muscle cells and mucous glands carried through the vagus nerve).

Asthma can further include acute episodes or attacks of additional airway narrowing via contraction of hyper-responsive airway smooth muscle that significantly increases airflow resistance. Asthma symptoms include recurrent episodes of breathlessness (e.g., shortness of breath or dyspnea), wheezing, chest tightness, and coughing. Additionally, COPD, often referred to as emphysema, is characterized by the alteration of lung tissue surrounding or adjacent to the airways in the lungs. Emphysema can involve destruction of lung tissue (e.g., alveolar sacs) that leads to reduced gas exchange and reduced circumferential traction applied to the airway wall by the surrounding lung tissue. The destruction of alveoli tissue restricts the in-flow of oxygen rich air and the proper function of healthier tissue, resulting in significant breathlessness. Exposure to chemicals or other substances (e.g., tobacco smoke) may significantly accelerate the rate of tissue damage or destruction. Additionally, chronic bronchitis, another type of COPD, is characterized by contraction of the airway smooth muscle, smooth muscle hypertrophy, excessive mucus production, mucous gland hypertrophy, and inflammation of airway walls. Like asthma, these abnormalities are the result of a complex interplay of local inflammatory cytokines, inhaled irritants, systemic hormones, local nervous system, and the central nervous system. Unlike asthma where respiratory obstruction may be largely reversible, the airway obstruction in chronic bronchitis is primarily chronic and permanent.

Treatment for pulmonary diseases includes reducing exposure to harmful agents, administering medications (e.g., bronchodilators, steroids, phosphodiesterase inhibitors, theophylline, antibiotics, etc.), administering lung therapy (e.g., oxygen therapy, pulmonary rehabilitation), and surgical intervention, such as bronchial thermoplasty. Unfortunately, pharmacological treatment requires patient compliance, often causes harmful side effects, and does not necessarily treat the underlying cause of the disease. Similarly, surgical intervention can result in the destruction of smooth muscle tone and nerve function, such that the patient is unable to respond favorably to inhaled irritants, systemic hormones, and both local and central nervous system input.

An alternative method for treating pulmonary disease is referred to as targeted lung denervation (TLD). This method utilizes ablation, such as monopolar or biopolar radiofrequency (RF) ablation, via an ablation assembly to selectively treat target regions inside of the airway wall (e.g., anatomical features in the stromas) while protecting the superficial tissues, such as the surface of the airway wall. For example, the mucous glands can be damaged to reduce mucus production a sufficient amount to prevent the accumulation of mucus that causes increased air flow resistance while preserving enough mucus production to maintain effective mucociliary transport, if needed or desired. Nerve branches/fibers passing through the airway wall or other anatomical features in the airway wall can also be destroyed.

Specially designed catheters allow for the introduction of an ablation assembly, generally comprising one or more collapsible electrodes or energy emitters, coupled to an expandable member, such as a balloon, into the airway of a patient via a delivery device. Once positioned in the desired region of the airway, such as the left and/or right main bronchi, the expandable member is expanded to position the one or more electrodes in contact with the airway wall. Energy, such as RF energy, is supplied to the energy emitter to ablate the targeted tissue, causing a lesion to form, therefore temporarily or permanently damaging the targeted tissue, therefore affecting, e.g. attenuating nerve signals to or from, portions of the lungs associated with the targeted tissue. Simultaneously, a coolant is supplied through the catheter and is directed to the one or more electrodes and into the expandable member or balloon. This allows for cooling of the superficial tissue in contact with the electrode, as well as the adjacent tissues. The size, shape, and depth of the lesions are determined by the flow rate and temperature of the coolant, and the energy supplied to the energy emitter(s). Devices, systems, and methods of such procedures can be found, for example, in one or more of U.S. Pat. No. 8,088,127 entitled “Systems, Assemblies, and Methods for Treating a Bronchial Tree,” and U.S. Pat. No. 9,649,153 entitled “Delivery Devices with Coolable Energy Emitting Assemblies,” both of which are incorporated herein by reference in their entireties.

In order to ensure that most or all of the target nerves extending along the airway are treated, it is generally desirable to form a circumferential lesion around all or most of the circumference of the airway wall. Due to design constraints or preferences, the electrode or energy emitter may not extend around the entirety of the circumference of the airway wall. Therefore, a circumferential lesion may be formed by ablating tissue while slowly rotating the ablation assembly or by positioning the ablation assembly in a series of rotational positions at each of which energy is delivered for a desired time period. The adjacent lesions then become contiguous and form a circumferential band all the way around the airway wall. Additionally or alternatively, the catheter may be repositioned axially to treat other locations within the airway distally or proximally of the first treatment site.

With respect to monopolar RF ablation systems, the systems generally include two separate monopolar electrodes, the active electrode (i.e., the energy emitter of the ablation assembly) and the dispersive electrode (e.g., a pad), which in combination with the patient's body, completes a circuit. The active electrode is designed to focus the current or power on the therapeutic target thereby creating a desired tissue effect, such as ablation. The dispersive electrode is positioned on the patent in a location remote from the surgical site and is relatively large in surface area, a design that serves to defocus or disperse the current thereby preventing or reducing the occurrence of non-target tissue injury. However, the variability of the electrical properties of the patient tissue interposed between the two electrodes of a monopolar system affects the consistency of the therapeutic effect.

Over the years various technologies have been developed to localize and guide tools or catheters through the bronchial pathways of the long. For example, conventional techniques may involve or include a bronchoscope or an endoscope and can include one or more viewing devices, such as optical viewing devices (e.g., cameras), optical trains (e.g., a set of lens), optical fibers, CCD chips, and the like. More complex visualization systems may involve or include fluoroscopy (e.g., the showing of a continuous x-ray image of the lungs while the procedure is performed). The general consensus has been that the increased risk of exposure to radiation through more complex visualization systems (e.g., involving fluoroscopy) is not justified during treatments in the proximal airways (e.g., in the bronchi where targeted lung denervation is typically performed). Nevertheless, three-dimensional visualization of the patient's airway would be helpful in pre-case planning to enable the physician to plan/review his or her treatment strategy before the procedure is performed, thereby increasing physician confidence and enabling an expedited procedure.

The present disclosure addresses these concerns.

### SUMMARY OF THE DISCLOSURE

The techniques of this disclosure generally relate to the use of sensors and navigation technology to standardize and decrease the complexity of monopolar or bipolar radiofrequency (RF) ablation in the treatment of target regions within an airway wall, thereby enabling the ablation of attenuating nerve signals to or from the targeted regions (sometimes referred to as “targeted lung denervation”). In some embodiments, using a virtual, three-dimensional (3-D) bronchial map from a recent computed tomography (CT) or magnetic resonance imaging (MRI) chest scan, in conjunction with positional sensing of an ablation tip of an expandable, cooled radiofrequency catheter, can enable detailed preplanning, as well as the ability to track the navigation of the catheter through different airways leading to and within the lungs of the patient, thereby increasing physician confidence, enabling an expedited procedure and optimizing procedure outcomes.

Moreover, merging a 3-D graphical rendering of the airway with real-time tracking of the catheter ablation tip within the airway space during the procedure can serve as an aid in precise placement of the ablation tip. For example, a digital representation of the electrode in real time, which is responsive to catheter manipulation, can enhance the facility of catheter adjustment for appropriate positioning by the practitioner, thereby decreasing the amount of time and experience needed to gain proficiency with the device and procedure. In some embodiments, sensors and navigational technology as disclosed herein can be used in conjunction with bronchoscopic and/or fluoroscopic imaging to provide improved visualization before, during and after the procedure. In yet other embodiments, the sensors and navigational technology can be used in place of conventional imaging techniques (e.g., eliminating the need for x-ray fluoroscopy).

In some embodiments, virtual markers or waypoints can be positioned at sites of interest within the airway (e.g., near the branch airways), and can be configured to provide real-time, potentially automated feedback during the procedure. For example, in some embodiments, the markers can provide guidance as to positioning of the catheter within the airway (e.g., provide an alert or automatically cease ablation when the ablation tip is positioned in proximity to a sensitive area), thereby improving safety and the likelihood of a successful procedure. In some embodiments, the markers can be virtually positioned on the 3-D graphical rendering, prior to the physical procedure being performed, thereby serving as a series of waypoints or checkpoints to ensure proper distal electrode placement, appropriate separation from side branch airways and other sensitive areas, appropriate rotational position, appropriate esophageal balloon location/distance, etc., thereby decreasing the amount of time needed for physicians to gain proficiency with the procedure, decreased procedure ambiguity, and increase the consistency of safe and effective outcomes.

In some embodiments, the sensors and navigational technology can be used to automate guidance and adjust an intensity of the ablation during the procedure. That is, understanding a 3-D location of the esophageal balloon relative to the ablation tip during the procedure can enable a processing unit to automatically adjust the power intensity of the ablation, thereby enabling a granular adjustment of power based on an exact distance between the ablation tip and the surface of the esophageal balloon thereby enabling greater precision in the depth of the ablation.

One embodiment of the present disclosure provides a pulmonary treatment system for treatment of target tissue in an airway of a subject, including a catheter assembly comprising a catheter shaft and an ablation tip or assembly located on a distal end of the catheter shaft. The ablation tip can include an expandable member, at least one energy emitter, and a cooling member, wherein the ablation tip is configured to be positioned within the airway of the subject such that expansion of the expandable member enables the at least one energy emitter and cooling member to engage a wall of the airway, the cooling member configured to cool a portion of the surface of the wall of the airway to reduce damage to the airway disposed between the at least one energy emitter and the target tissue, while the at least one energy emitter delivers energy to the target tissue to create one or more nerve attenuating lesions, and a control assembly including a display configured to depict a three-dimensional graphical rendering of the airway of the subject for preplanning prior to the procedure, with real-time tracking of the ablation tip during the treatment of the target tissue.

In one embodiment, the system further includes an electromagnetic field generator configured to generate an electromagnetic field sufficient to surround a portion of the subject. In one embodiment, the system further includes one or more sensors positioned on the catheter assembly, such as on the ablation tip on the expandable member, energy emitter, optional Nitinol wire extending within the expandable member, catheter shaft, or any combination thereof. The one or more sensors are configured to sense one or more parameters of interest. In one embodiment, the sensor is configured to determine a position of the at least one energy emitter within a three-dimensional geometric coordinate system established by an electromagnetic field generator. In one embodiment, the sensor is further configured to monitor at least one of a temperature, inflation pressure, coolant flow rate, tissue impedance, current, or power associated with the ablation tip.

In one embodiment, the control assembly is configured to plot a determined position of the ablation tip in real-time on the three-dimensional graphical rendering of the airway of the subject. In one embodiment, the system further includes one or more reference datum selectively positionable on the subject and configured to follow movements of the subject while inhaling and exhaling, thereby enabling compensation of a determined position of the ablation tip within the three-dimensional graphical rendering of the airway of the subject.

In one embodiment, the control assembly is configured to enable one or more virtual waypoints to be positioned within the three-dimensional graphical rendering of the airway of the subject, thereby enabling preplanning of ablation tip placement within the airway of the subject prior to treatment. In one embodiment, the control assembly is further configured to limit electrical power to the at least one energy emitter until a sensed position of the ablation tip corresponds with the one or more virtual waypoints. In one embodiment, the control system is further configured to automatically provide an indication that the preplanned procedure has been completed at the one or more virtual waypoints during the treatment.

In one embodiment, the one or more sensors is coupled to the energy emitter, such as an electrode, and the location of the energy emitter is determined based on the location of the one or more sensors. In another embodiment, the one or more sensors is coupled to the ablation tip proximate the energy emitter, such as on or in the expandable member, catheter shaft, or Nitinol wire extending within the expandable member, but not on the energy emitter, and a 3-D offset is computed, i.e. the distance from the energy emitter to the sensor. This allows the location of the energy emitter in space to be determined because a known or consistent offset has been computed.

In yet another embodiment, one or more sensors are coupled to the catheter assembly as described above, and one or more sensors are coupled to an assembly or device positioned outside of the airway, such as an esophageal cooling assembly placed in the esophagus. The control assembly is configured to calculate a distance between the sensors, which can then be used to control the power delivered to the energy emitter of the ablation tip based on the determined distance. For example, if the determined distance falls below a predetermined threshold, e.g. the energy emitter is close to the esophagus, the control assembly automatically lowers the power delivery to the energy emitter, and/or can shut off power delivery altogether if a critical distance is reached. This replaces the need for the physician to manually alter the level of power delivery based on proximity to the esophagus or any other heat sensitive tissue marked by another sensor, which ultimately increases the safety of the catheter assembly and the methods of treatment performed using the catheter assembly.

Another embodiment of the present disclosure provides a method for pulmonary treatment of target tissue in an airway of a subject, including positioning an ablation tip of a catheter assembly according to any of the embodiments described above within the airway of the subject, the ablation tip including an expandable member, at least one energy emitter, and a cooling member, wherein the expandable member enables the at least one energy emitter and cooling member to engage a wall of the airway, the cooling member configured to cool a portion of the surface of the wall of the airway to reduce damage to the airway disposed between the at least one energy emitter and the target tissue, while the at least one energy emitter delivers energy to the target tissue to create one or more nerve attenuating lesions; displaying a three-dimensional graphical rendering of the airway of the subject for preplanning prior to the procedure; and tracking of the ablation tip during the treatment of the target tissue. Optionally, a control system can automatically regulate the power delivery to the energy emitter(s) of the ablation tip during treatment based on a relative position of the ablation tip or proximity of the ablation tip to sensitive non-target tissue. For example, if an ablation tip is determined to be below a threshold distance from the esophagus or other heat sensitive tissue, the power level is lowered, or in certain circumstances, shut off completely, to avoid unnecessary ablation or damage to sensitive non-target tissues.

Yet another embodiment of the present disclosure provides a pulmonary treatment system for treatment of target tissue in an airway of a subject, including a catheter assembly comprising an ablation tip including an expandable member, at least one energy emitter, and a cooling member, wherein the ablation tip is configured to be positioned within the airway of the subject such that expansion of the expandable member enables the at least one energy emitter and cooling member to engage a wall of the airway, the cooling member configured to cool a portion of the surface of the wall of the airway to reduce damage to the airway disposed between the at least one energy emitter and the target tissue, while the at least one energy emitter delivers energy to the target tissue to create one or more nerve attenuating lesions, an electromagnetic field generator configured to generate an electromagnetic field sufficient to surround a portion of the subject, a sensor positioned on or proximate to the ablation tip (e.g. on or in the energy emitter, catheter shaft, Nitinol wire, expandable member, or combinations thereof, etc.) configured to determine a position of the at least one energy emitter within a three-dimensional geometric coordinate system established by the electromagnetic field generator, and a control assembly including a display configured to depict a three-dimensional graphical rendering of the airway of the subject for preplanning prior to the procedure, and to plot a determined position of the ablation tip in real-time on the three-dimensional graphical rendering of the airway of the subject during the treatment of the target tissue, wherein the control assembly is further configured to enable one or more virtual waypoints to be positioned within the three-dimensional graphical rendering of the airway of the subject, thereby enabling preplanning of ablation tip placement within the airway of the subject prior to treatment, wherein the control assembly is further configured to limit electrical power to the at least one energy emitter until a sensed position of the ablation tip corresponds with the one or more virtual waypoints, and wherein the control system is further configured to automatically provide an indication that preplanned procedure has been completed at the one or more virtual waypoints during the treatment.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1A is a perspective view of a lung denervation system for ablating target tissue of an airway of a patient during treatment graphical rendering of the airway, in accordance with an embodiment of the disclosure.

FIG. 1B is a close-up view of an ablation tip of the system of FIG. 1A, in accordance with an embodiment of the disclosure.

FIG. 2 is a schematic view depicting a lung denervation system in use on a patient during treatment, in accordance with an embodiment of the disclosure.

FIG. 3 is a three-dimensional graphical rendering of a portion of the lungs of the patient including one or more virtual waypoints or checkpoints, in accordance with an embodiment of the disclosure.

FIG. 4A is a three-dimensional graphical rendering of a portion of the lungs of the patient prior to performance of the procedure, in accordance with an embodiment of the disclosure.

FIG. 4B is a three-dimensional graphical rendering of a portion of the lungs of the patient after performance of the procedure, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

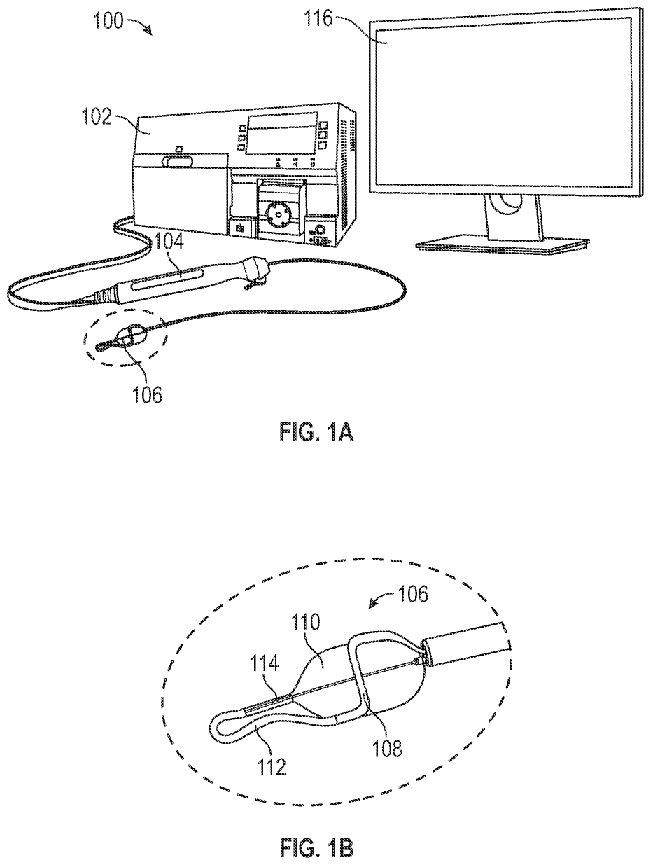

Referring to FIG. 1A, a lung denervation system 100 for ablating target tissue of an airway during treatment of a patient. In embodiments, the system can be configured to enable merging a 3-D graphical rendering of the airway with real-time tracking of a catheter ablation tip within the airway during the procedure is depicted in accordance with an embodiment of the disclosure. In embodiments, the system 100 can include a control assembly 102 (occasionally referred to as a “console”), which can include a display 116, operably coupled to an ablation catheter assembly 104 (occasionally referred to as a “dual-cooled radiofrequency catheter”).

With additional reference to FIG. 1B, the ablation catheter assembly 104 can include an ablation tip 106 comprising one or more energy emitters 108, such as an electrode or transducer, and an expandable member 110 (alternatively referred to as an “esophageal balloon”). One or more energy emitters 108 are configured to delivery energy in the form of RF, microwave, or ultrasound, for example. Accordingly, the system 100 can be configured to deliver radio frequency energy to modulate or disable the pulmonary plexus. The RF energy can be delivered to different target regions, which can include, without limitation, nerve tissue (e.g., tissue of the vagus nerves, nerve trunks, etc.), fibrous tissue, diseased or abnormal tissues (e.g., cancerous tissue, inflamed tissue, and the like), cardiac tissue, muscle tissue, blood, blood vessels, anatomical features (e.g., membranes, glands, cilia, and the like), or other sites of interest. While embodiments shown are configured for delivery of RF energy, other configurations can be adapted to accommodate a catheter based microwave antenna, high energy pulse electroporation, or similar energy modalities.

In RF ablation, heat is generated due to the tissue resistance as RF electrical current travels through the tissue. The tissue resistance results in power dissipation that is equal to the current flow squared times the tissue resistance. To ablate deep tissues, tissue between an RF electrode and the deep tissue can become overheated if active cooling is not employed using a cooling device, such as a cooling plate or cooling balloon. Accordingly, in some embodiments, one or more energy emitters 108 can be coupled to a cooling member 112 configured for flowing coolant therethrough to cool energy emitters 108 to protect surface tissue in contact with energy emitter 108 and adjacent to energy emitter 108 to accomplish deep tissue ablation. Thus, the cooling member 112 can be used to keep tissue near the electrode below a temperature that results in cell death or damage, thereby protecting tissue. In some embodiments, the coolant and electrode produce a lesion at a therapeutic depth of at least about 3 mm while protecting tissue at shallower depths from lethal injury. In some embodiments, the lesions can be formed at a depth of about 3 mm to about 5 mm to damage nerve tissue; although other temperatures and depths can be achieved.

In embodiments, the ablation catheter assembly 104 can further include one or more sensors 114 to provide positional data of the ablation tip 106, as well as to monitor other parameters of interest (e.g., temperature, inflation pressure, coolant flow rate, tissue impedance, current, power, etc.). Feedback from the one or more sensors 114 can be used to depict a position of the ablation tip 106 within the airway of the patient, as well as to modulate the power and/or current delivered to electrode(s). In some embodiments, outputted energy can be adjusted to account for local variations in tissue that alters the local impedance, thus avoiding excess heating which can lead to unwanted hot spots.

Accordingly, during operation, a bronchoscope can be passed into the patient's mouth and into the lungs, thereafter a portion of the catheter assembly 104 can be passed through the bronchoscope, thereby enabling the ablation tip 106 to be positioned within the airway to provide treatment. Once the ablation tip 106 is in place, the expandable member 110 can be inflated, thereby enabling the energy emitter 108 and cooling member 112 to engage wall of the airway. The cooling member 112 can be configured to cool portion of the surface of the wall of the airway to reduce damage to the airway disposed between the at least one energy emitter and the target tissue, while the energy emitter 108 delivers radiofrequency energy which penetrates to interrupt the nerves just outside of the airways. radiofrequency energy which penetrates to interrupt the nerves just outside of the airways. Once the energy has been delivered in one position, the balloon can be deflated and moved and/or rotated to the next position, upon completion, the catheter assembly 104 is removed. Following treatment, the nerves distal to the treatment site are interrupted, thereby decreasing nerve signals throughout the lung on that side. The catheter 104 is then placed in the lung on the opposite side and the treatment is continued. The catheter 104 and bronchoscope can then be removed from the patient.

With continued reference to FIG. 1A, to promote enhanced visualization and navigation, in some embodiments, the control assembly 102 can include an electronic control unit including a computer processing unit (CPU) and a monitor or display 116 configured to depict a 3-D graphical rendering of the lungs and/or airway of the patient for preplanning prior to the procedure, with real-time tracking of the ablation tip 106 during the procedure. In embodiments, real-time tracking of the ablation tip 106 within the 3-D graphical rendering during the procedure can serve as an aid in precise placement of the ablation tip 106. For example, a digital representation of energy emitter 106 in real time, which is responsive to catheter 104 manipulation, can enhance the facility of catheter 104 adjustment for appropriate positioning by the practitioner, thereby decreasing the amount of time and experience needed to gain proficiency with the system and procedure.

Throughout this disclosure, the terms disrupt, ablate, modulate, denervate will be used. It should be understood that these terms globally refer to any manipulation of the nerve that changes the action of that nerve. In embodiments, manipulation can be a total cessation of signals, as in ablation or severing, or manipulation can be a modulation, as is done by partial or temporary disruption, pacing, etc. Similarly, trachea is often used to describe a segment wherein the devices and methods will be used. It should be understood that this is shorthand and can be meant to encompass the trachea itself, as well as the right and left main bronchi and other portions of the pulmonary tree as necessary.

It should be noted that the pulmonary nerves referred to in the disclosure not only include nerves that innervate the pulmonary system but also any neural structures that can influence pulmonary behavior. For example, elements of the cardiac plexus, or the nerves that innervate the esophagus, also interact with the airways and may contribute to asthmatic conditions. The nerves can include nerve trunks along the outer walls of hollow vessels, nerve fibers within the walls of hollow vessels (e.g., the wall of the trachea and/or esophagus), nerves within a bridge between the trachea and esophagus, or at other locations. The left and right vagus nerves originate in the brainstem, pass through the neck, and descend through the chest on either side of the trachea. These or a portion of these nerves can be targeted. The vagus nerves spread out into nerve trunks that include the anterior and posterior pulmonary plexuses that wrap around the trachea, the left main bronchus, and the right main bronchus. The nerve trunks also extend along and outside of the branching airways of the bronchial tree. Nerve trunks are the main stem of a nerve comprising a bundle of nerve fibers bound together by a tough sheath of connective tissue. The vagus nerves, including their nerve trunks, along the trachea or other nerve tissue along, proximate to, or in the bronchial tree can be targeted, while branches that run along, proximate to, or in the esophagus can be not targeted and/or protected via the embodiments set forth below. A treatment device in the form of a system 100 for ablating targeted tissue can be positioned at different locations within an airway (e.g., the trachea, one of the main stem bronchi, or other structures of the bronchial tree).

The pulmonary branches of the vagus nerve along the left and right main stem, and bronchus intermedius are particularly preferred targets. The nerve trunks of the pulmonary branches extend along and outside of the left and right main stem bronchus and distal airways of the bronchial tree. Nerve trunks of the main stem nerve comprise a bundle of nerve fibers bound together by a tough sheath of connective tissue. Any number of procedures can be performed on one or more nerve trunks to affect the portion of the lung associated with those nerve trunks. Because some of the nerve tissue in the network of nerve trunks coalesce into other nerves (e.g., nerves connected to the esophagus, nerves though the chest and into the abdomen, and the like), specific sites can be targeted to minimize, limit, or substantially eliminate unwanted damage of those other nerves. Some fibers of anterior and posterior pulmonary plexuses coalesce into small nerve trunks which extend along the outer surfaces of the trachea and the branching bronchi and bronchioles as they travel outward into the lungs. Along the branching bronchi, these small nerve trunks continually ramify with each other and send fibers into the walls of the airways. Any of those nerve trunks or nerve tissue in walls can be targeted.

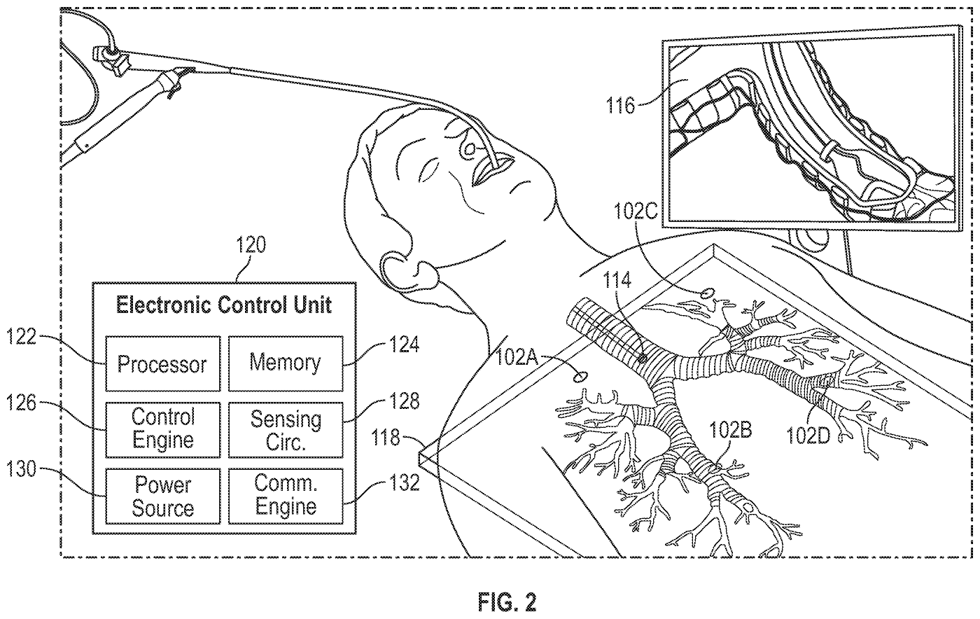

With additional reference to FIG. 2, a schematic view depicting a system 100 configured to enable detailed preplanning for a tissue ablation procedure, as well as the ability to track the navigation a catheter assembly 104 for precise positioning of an ablation tip 106 during the procedure, is depicted in accordance with an embodiment of the disclosure. As depicted, the patient can be positioned on an electromagnetic field generator 118. For example, in some embodiments, the electromagnetic field generator 118 can be configured to provide a flat surface for the patient to lie down, wherein the electromagnetic field generator generates electromagnetic field sufficient to surround a portion of the patient. The sensor 114 positioned on the ablation tip 106 is used to determine a location of the energy emitter 108 within the electromagnetic field generated by the electromagnetic field generator 118 (e.g., determine x-, y-, z-coordinates of the energy emitter 108 within a three-dimensional geometric coordinate system established by the electromagnetic field generator 118).

Accordingly, in some embodiments, the sensor 114 is an electromagnetic field sensor configured to sense an electromagnetic field generated by the electromagnetic field generator 118. The sensed electromagnetic field can be used to identify a location of the electromagnetic field sensor 114 in accordance with a coordinate system of the electromagnetic field. Thus, a sensed electromagnetic field received by the electromagnetic field sensor 114 can be communicated to the control assembly 102 (e.g., including an electronic control unit 120), where a precise location of the ablation tip 106 within the coordinate system of the electromagnetic field generated by the electromagnetic field generator 118 can be determined. In some embodiments, the control assembly 102 can compare a location of the electromagnetic field sensor 114 with the 3-D graphical rendering of the airway, so as to merge the determined position of the ablation tip 106 with the 3-D graphical rendering of the airway shown on the display 116.

In some embodiments, to track a specific location of the electromagnetic field sensor 114 within the system may incorporate reference to a predefined map, such as a Computed Tomography (CT) scan. For example, in such embodiments, a CT scan may be used in accordance with its image processing software capabilities to analyze real time images from the catheter 104 and bronchoscope and compare the captured images to expected images from the CT scan. This can be commonly known as fiber optic shape sensing.

In some embodiments, the electromagnetic field generator 118 can be configured to be operably coupled with one or more reference datum 120A-D, which can be selectively positioned on the chest of a patient. The one or more reference datum 120A-D can be configured to move up and down following chest movements of the patient while inhaling and exhaling. Accordingly, in embodiments, movement of the reference sensors in the electromagnetic field can be captured by the one or more reference datum 120A-D and transmitted to the control assembly 120, so that a breathing pattern of the patient can be observed in real time. In this manner, a location of the electromagnetic field sensor 114 can be compensated for so that the compensated location of the ablation tip 106 is synchronized with the 3-D graphical rendering of the airway. Thereafter, the precise position of the ablation tip 106 relative to the 3-D graphical rendering of the airway can be depicted on the display 116.

Alternately, in some embodiments, the electromagnetic field generator 118 can be configured to be operable through user navigation. For example, a user navigates to a specific location or locations within an airway (such as, for example, main carina and secondary carinas) such that a corresponding CPU can reconcile the actual location of the electrode tip at the user navigated specific location or locations in real space with the reconstructed location in the graphical rendering. In such embodiments, there would be no requirement for external reference datum 120A-D as the user would navigate and select the specific location or locations.

Sensor(s) 114 can be configured throughout various aspects of the system, including at least at or near the RF electrode, including at least a Nitinol wire extending through the shaft and into the expandable member, the expandable member, and/or catheter 104 shaft. In some embodiments, sensor 114 can be configured such that it is located away from the RF electrode if a consistent or known offset from the distance of the RF electrode to the actual location is available. In such instances to compensate for the location offset from the RF electrode a 3-D offset of the location of the sensor 114 can be computed based on the rotation of the sensor 114.

Additionally or alternately, the system can be configured to compute distances between disparate sensors. Beneficially, allowing computation of distances between disparate sensors would allow physicians and medical professionals more time to devote to diagnostics and patient care instead of requiring manual alteration of power level for power delivery based on proximity of each disparate sensor to heat sensitive tissues or organs. For example, ability to compute distances between the catheter assembly as described herein, and one or more sensors placed outside of the airway, such as on an esophageal cooling catheter positioned within or around the esophagus or other heat sensitive tissues marked with a sensor, would enable power delivery to be automatically adjusted by the ECU 120 based on a proximity of the ablation assembly to heat sensitive tissues. For example, the ECU 120 may lower power delivery to the energy emitter when a distance between sensor falls below a predetermined threshold, or may completely shut off power delivery if the distance falls below a critical threshold, without requiring manual input from the user of the catheter assembly. In turn, reducing or eliminating the need for human input would also reduce or eliminate potential for human error and thereby increase overall safety of the system.

In one embodiment, the ECU 120 or components thereof can comprise or include various modules or engines, each of which is constructed, programmed, configured, or otherwise adapted to autonomously carry out a function or set of functions. The term “engine” as used herein is defined as a real-world device, component, or arrangement of components implemented using hardware, such as by an application specific integrated circuit (ASIC) or field programmable gate array (FPGA), for example, or as a combination of hardware and software, such as by a microprocessor system and a set of program instructions that adapt the engine to implement the particular functionality, which (while being executed) transform the microprocessor system into a special-purpose device.

An engine can also be implemented as a combination of the two, with certain functions facilitated by hardware alone, and other functions facilitated by a combination of hardware and software. In certain implementations, at least a portion, and in some cases, all, of an engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques.

Accordingly, each engine can be realized in a variety of physically realizable configurations, and should generally not be limited to any particular implementation exemplified herein, unless such limitations are expressly called out. In addition, an engine can itself be composed of more than one sub-engines, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

In some embodiments, ECU 120 can include a processor 122, memory 124, a control engine 126, sensing circuitry 128, and a power source 130. Optionally, in embodiments, ECU 120 can further include a communications engine 132. Processor 122 can include fixed function circuitry and/or programmable processing circuitry. Processor 122 can include any one or more of a microprocessor, a controller, a DSP, an ASIC, an FPGA, or equivalent discrete or analog logic circuitry. In some examples, processor 122 can include multiple components, such as any combination of one or more microprocessors, one or more controllers, one or more DSPs, one or more ASICs, or one or more FPGAs, as well as other discrete or integrated logic circuitry. The functions attributed to processor 122 herein may be embodied as software, firmware, hardware or any combination thereof.

Memory 124 can include computer-readable instructions that, when executed by processor 122 cause ECU 120 to perform various functions. Memory 128 can include volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Control engine 126 can include instructions to control the components of ECU 120 and instructions to selectively control and applied power to the energy emitter 108. For example, based on conditions detected by the sensing circuitry 128 or sensor 114 (e.g. a position of the ablation tip 106 within the lungs of the patient, a distance between the energy emitter 106 and the expansion member 110, etc.), control engine 126 can adjust an energy output of the energy emitter 108, thereby limiting the degree of ablation permitted by the ablation tip 106.

In embodiments, sensing circuitry 128 can be configured to sense one or more signals received by the sensor 114, including a position of the sensor 114 within the electromagnetic field generated by the electromagnetic field generator 118, as well as other parameters of interest (e.g., temperature, inflation pressure, coolant flow rate, tissue impedance, current, power, etc.). Accordingly, sensing circuitry 128 can include or can be operable with the one or more sensors 114. In embodiments, sensing circuitry 114 can additionally include one or more filters and amplifiers for filtering and amplifying signals received from one or more sensors.

Power source 130 can be configured to deliver operating power to the components of ECU 120. Optionally, communications engine 132 can include any suitable hardware, firmware, software, or any combination thereof for communicating with other components of the system 100 and/or external devices. Under the control of a processor 122, communication engine 132 can receive downlink telemetry from, as well as send uplink telemetry to one or more external devices using an internal or external antenna. In addition, communication engine 132 can facilitate communication with a networked computing device and/or a computer network. For example, communications engine 132 can receive updates to instructions for control engine 126 from one or more external devices.

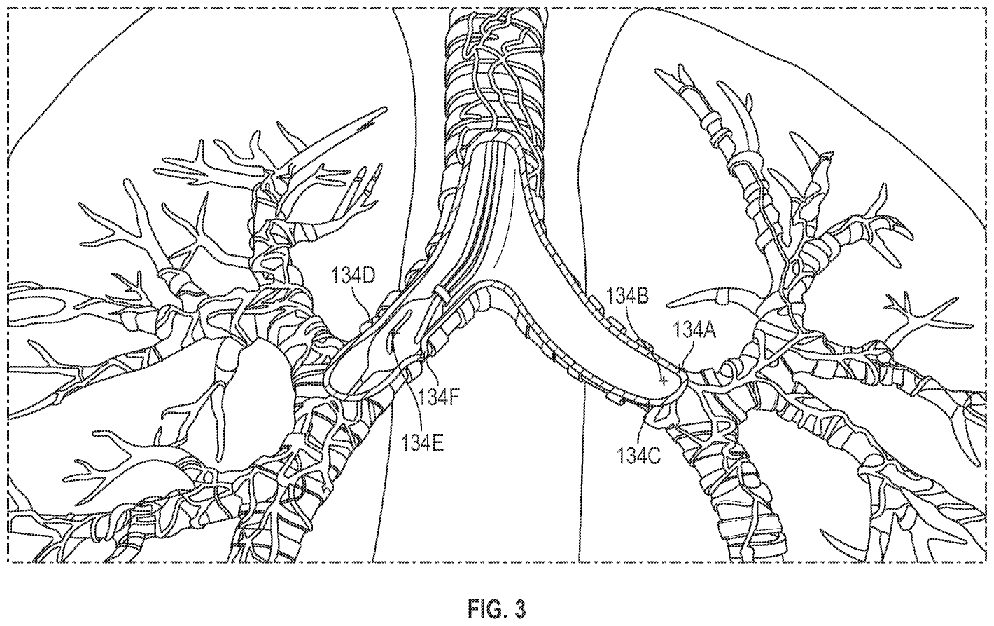

With additional reference to FIG. 3 a three-dimensional graphical rendering of a portion of the lungs of the patient is depicted in accordance with an embodiment of the disclosure. For example, in some embodiments, the three-dimensional graphical rendering can be based on a recent CT or MRI chest scan, thereby enabling a clinician to preplan the procedure prior to commencement. As an aid in preplanning, in some embodiments, one or more virtual markers 134A-F can be positioned electronically at sites of interest within the airway (e.g., near the branch airways). Thereafter, the one or more virtual markers 134A-F can provide real-time, potentially automated feedback and control of the energy emitter 108 during the procedure.

For example, in some embodiments, the markers 134A-F can provide guidance as to positioning of the ablation tip 106 within the airway (e.g., provide an alert or automatically cease ablation when the ablation tip is positioned in proximity to a sensitive area), thereby improving safety and the likelihood of a successful procedure. In some embodiments, the markers 134A-F can be virtually positioned on the 3-D graphical rendering, prior to the physical procedure being performed, thereby serving as a series of waypoints or checkpoints to ensure proper distal electrode placement, appropriate separation from side branch airways and other sensitive areas, appropriate rotational position, appropriate esophageal balloon location/distance, etc., thereby decreasing the amount of time needed for physicians to gain proficiency with the procedure, decreased procedure ambiguity, and increase the consistency of safe and effective outcomes. In some embodiments, the markers 134A-F can be configured to change color or provide another indication of a fulfillment of the procedure at the desired waypoints.

In some embodiments, the sensors and navigational technology can be used to automate guidance and adjust an intensity of the ablation during the procedure. That is, understanding a 3-D location of the expandable member 110 relative to the energy emitter 108 during the procedure can enable a processing unit to automatically adjust the power intensity of the ablation, thereby enabling a granular adjustment of power based on an exact distance between the ablation tip 106 and the surface of the expandable member 110 thereby enabling greater precision in the depth of the ablation. To facilitate measurement of a distance between the surface of the expandable member 110 and the energy emitter 108, in some embodiments, both the expandable member 110 and the energy emitter 108 can include sensors 114.

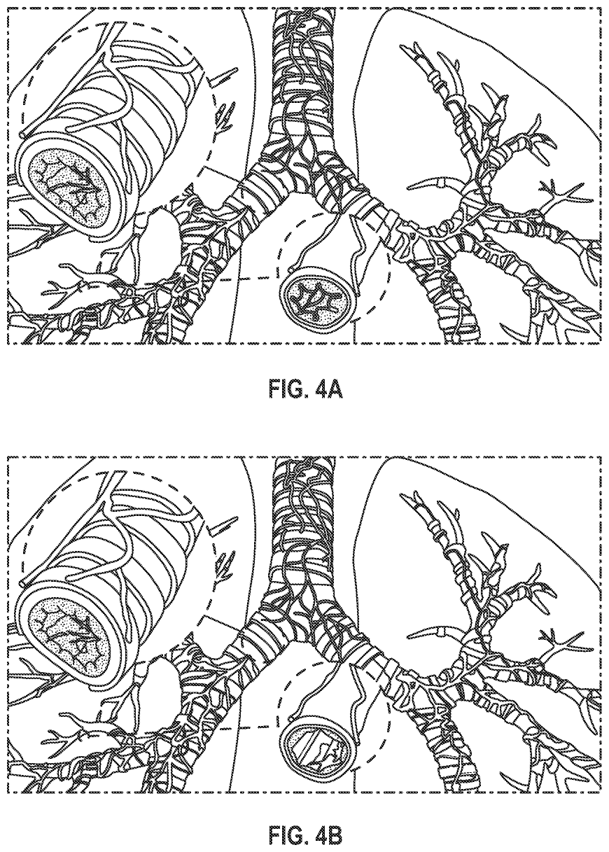

With additional reference to FIGS. 4A-B, in some embodiments the system 100 can further be configured to compare a three-dimensional graphical rendering of at least a portion of the lungs of the patient prior to performance of the procedure (as depicted in FIG. 4A) with a three-dimensional graphical rendering of at least a portion of the lungs of the patient after performance of the procedure (as depicted in FIG. 4B), thereby depicting a measurable relaxation and/or opening of the airways after the procedure. Data gathered by the system 100, including specific locations of ablation within the patient's lungs and the degree to which the airways have opened up (as well as potential other factors, such as patient comfort and other symptoms) can be used as an aid in determining future locations for desired ablations, potentially in other patients.

Accordingly, embodiments of the present disclosure provide a system for ablating target tissue 100 configured to display a virtual, three-dimensional of at least a portion of the lungs of a patient, in conjunction with positional sensing of an ablation tip 106 of a targeted lung denervation catheter 104, thereby enabling detailed preplanning of the ablation procedure, as well as the ability to track the navigation of the ablation tip 106 through airway, thereby increasing physician confidence and enabling an expedited procedure.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35

1. A pulmonary treatment system for treatment of target tissue in an airway of a subject, the system comprising:
a catheter assembly comprising an ablation assembly including an expandable member, at least one energy emitter, and a cooling member, wherein the ablation assembly is configured to be positioned within the airway of the subject such that expansion of the expandable member enables the at least one energy emitter and cooling member to engage a wall of the airway, the cooling member configured to cool a portion of the surface of the wall of the airway to reduce damage to the airway disposed between the at least one energy emitter and the target tissue, while the at least one energy emitter delivers energy to the target tissue to create one or more nerve attenuating lesions; and
a control assembly a display, the control assembly being configured to display a three-dimensional graphical rendering of the airway of the subject for real-time tracking of the ablation assembly during the treatment of the target tissue.

2. The system of claim 1, further comprising an electromagnetic field generator configured to generate an electromagnetic field sufficient to surround a portion of the subject.

3. The system of claim 1, wherein the three-dimensional graphical rendering of the airway comprises a predefined map generated by a computed tomography scan (CT scan), and wherein the real-time tracking of the ablation assembly is generated by a comparison of the predefined map and real time imaging generated by a bronchoscope or other imaging assembly introduced into the airway during treatment.

4. The system of claim 1, further comprising a sensor positioned on the catheter assembly configured to sense one or more parameters of the airway and/or the ablation assembly.

5. The system of claim 4, wherein the sensor is positioned on the at least one energy emitter, the sensor being configured to determine a position of the at least one energy emitter within a three-dimensional geometric coordinate system.

6. The system of claim 4, wherein the sensor is positioned offset the at least one energy emitter at an offset distance, the sensor being configured to determine a position of the at least one energy emitter within a three-dimensional geometric coordinate system using the offset distance.

7. The system of claim 4, wherein the sensor is further configured to monitor at least one of a temperature, inflation pressure, coolant flow rate, tissue impedance, current, or power associated with the ablation assembly.

8. The system of claim 1, wherein the control assembly is configured to plot a determined position of the ablation assembly in real-time on the three-dimensional graphical rendering of the airway of the subject.

9. The system of claim 1, further comprising one or more reference datum selectively positionable on the subject and configured to follow movements of the subject while inhaling and exhaling, thereby enabling compensation of a determined position of the ablation assembly within the three-dimensional graphical rendering of the airway of the subject.

10. The system of claim 1, wherein the control assembly is configured to enable one or more virtual waypoints to be positioned within the three-dimensional graphical rendering of the airway of the subject, thereby enabling preplanning of ablation assembly placement within the airway of the subject prior to treatment.

11. The system of claim 10, wherein the control assembly is further configured to limit electrical power to the at least one energy emitter until a sensed position of the ablation assembly corresponds with the one or more virtual waypoints.

12. The system of claim 10, wherein the control system is further configured to automatically provide an indication that a preplanned procedure has been completed at the one or more virtual waypoints during the treatment.

13. A method for pulmonary treatment of target tissue in an airway of a subject, the method comprising:
positioning an ablation assembly of a catheter assembly within the airway of the subject, the ablation assembly including an expandable member, at least one energy emitter, and a cooling member, wherein the expandable member enables the at least one energy emitter and cooling member to engage a wall of the airway, the cooling member configured to cool a portion of the surface of the wall of the airway to reduce damage to the airway disposed between the at least one energy emitter and the target tissue, while the at least one energy emitter delivers energy to the target tissue to create one or more nerve attenuating lesions;
displaying a three-dimensional graphical rendering of the airway of the subject for preplanning prior to the procedure; and
tracking a position of the ablation assembly during the treatment of the target tissue.

14. The method of claim 13, further comprising generating an electromagnetic field sufficient to surround a portion of the subject.

15. The method of claim 13, further comprising sensing one or more parameters of interest in proximity to the ablation assembly.

16. The method of claim 13, further comprising determining a position of the at least one energy emitter within a three-dimensional geometric coordinate system established by an electromagnetic field generator.

17. The method of claim 16, further comprising controlling a level of power delivery to the at least one energy emitter based on the position of the at least one energy emitter.

18. The method of claim 17, wherein controlling the level of power delivery comprises one of lowering the level of power delivery or shutting off power delivery if the position of the ablation assembly is within a predetermined distance from a predetermined position of non-target tissue.

19. The method of claim 11, further comprising:
positioning a sensor outside of the airway;
determining in real-time a distance between the ablation assembly and the sensor outside of the airway; and
controlling a level of power delivery to the at least one energy emitter based on the position of the at least one energy emitter.

20. The method of claim 19, wherein controlling the level of power delivery comprises one of lowering the level of power delivery or shutting off power delivery if the distance falls below a predetermined threshold distance.

21. The method of claim 19, wherein positioning a sensor outside of the airway comprises positioning an esophageal assembly having the sensor thereon within an esophagus of the subject, and wherein the distance is a distance measured between the sensor of the esophageal assembly and a sensor coupled to the catheter assembly.

22. The method of claim 11, further comprising plotting a determined position of the ablation assembly in real-time on the three-dimensional graphical rendering of the airway of the subject.

23. The method of claim 11, further comprising using one or more reference datum to follow movements of the subject while inhaling and exhaling, thereby enabling compensation of a determined position of the ablation assembly within the three-dimensional graphical rendering of the airway of the subject.

24. The method of claim 11, further comprising positioning one or more virtual waypoints within the three-dimensional graphical rendering of the airway of the subject, thereby enabling preplanning of ablation assembly placement within the airway of the subject prior to treatment.

25. The method of claim 23, further comprising limiting electrical power to the at least one energy emitter until a sensed position of the ablation assembly corresponds with the one or more virtual waypoints.

26. The method of claim 23, further comprising providing an indication that a preplanned procedure has been completed at the one or more virtual waypoints during the treatment.

27. A pulmonary treatment system for treatment of target tissue in an airway of a subject, the system comprising:
a catheter assembly comprising an ablation assembly including an expandable member, at least one energy emitter, and a cooling member, wherein the ablation assembly is configured to be positioned within the airway of the subject such that expansion of the expandable member enables the at least one energy emitter and cooling member to engage a wall of the airway, the cooling member configured to cool a portion of the surface of the wall of the airway to reduce damage to the airway disposed between the at least one energy emitter and the target tissue, while the at least one energy emitter delivers energy to the target tissue to create one or more nerve attenuating lesions;
an electromagnetic field generator configured to generate an electromagnetic field sufficient to surround a portion of the subject;
a sensor positioned on the catheter assembly, the sensor being configured to determine a position of the at least one energy emitter within a three-dimensional geometric coordinate system established by the electromagnetic field generator; and
a control assembly including a display configured to depict a three-dimensional graphical rendering of the airway of the subject for preplanning prior to the procedure, and to plot a determined position of the ablation assembly in real-time on the three-dimensional graphical rendering of the airway of the subject during the treatment of the target tissue, wherein the control assembly is further configured to enable one or more virtual waypoints to be positioned within the three-dimensional graphical rendering of the airway of the subject, thereby enabling preplanning of ablation assembly placement within the airway of the subject prior to treatment, wherein the control assembly is further configured to limit electrical power to the at least one energy emitter until a sensed position of the ablation assembly corresponds with the one or more virtual waypoints, and wherein the control system is further configured to automatically provide an indication that preplanned procedure has been completed at the one or more virtual waypoints during the treatment.
